(19) United States
(12) Patent Application Publication
(10) Pub. No.: US 2019/0297821 A1
(43) Pub. Date: Oct. 3, 2019

(54) CROSS-HYBRIDIZATION OF DISTINCT HOMOZYGOUS CANNABIS PLANTS TO PRODUCE CONSISTENT EARLY FLOWERING SEEDS

(71) Applicant: JACK HEMPICINE LLC, Albany, OR (US)

(72) Inventors: Seth S. Crawford, Albany, OR (US); Eric B. Crawford, Corvallis, OR (US)

(21) Appl. No.: 16/364,715

(22) Filed: Mar. 26, 2019

Related U.S. Application Data
(60) Provisional application No. 62/649,424, filed on Mar. 28, 2018.

(51) Int. Cl. A01H 6/28; A01H 5/02; C12Q 1/6827; C12Q 1/6895

(52) U.S. Cl. CPC A01H 6/28; C12Q 1/6895; C12Q 1/6827; A01H 5/02

(57) ABSTRACT

A method for developing seeds for consistent early flowering short-day Cannabis plants, the method including crossbreeding a short-day Cannabis plant with a day-neutral Cannabis plant, and collecting the seeds produced through crossbreeding.

### CROSS REFERENCE TO RELATED APPLICATION

This application claims priority to and the benefit of U.S. Provisional Patent Application No. 62/649,424 filed Mar. 28, 2018, which is incorporated herein by reference in its entirety.

### TECHNICAL FIELD

This disclosure is directed to the production of seeds for early flowering Cannabis plants, and, more particularly, to a method of crossbreeding distinct homogenous species of photosensitive short-day Cannabis plants with day-neutral Cannabis plants to achieve a first generation of consistent early flowering Cannabis plants for optimizing field use and harvest yields.

### BACKGROUND

Cannabis is a genus of plants useful in the industrial or artisanal production of oil, fiber, food, fragrance, and medicine. The various parts of Cannabis plants may be used in a near infinite number of products, such as fiber, oils, and medicines, for example.

As the number of indoor Cannabis growing operations expands, increasing amounts of energy and electricity are required for the lighting needs of the plants. With further legalization, it is expected that prices for Cannabis products will lower, making it harder for indoor operations to afford the overhead for their power costs and further incentivizing the switch to natural, outdoor growing. However, in contrast to the continuously controlled lighting cycles of indoor operations, outdoor growing is subject to the seasonal timing of natural sunlight cycles. Typically, sun-grown Cannabis is planted in spring, flowers when nighttime exceeds about 10-12 hours, and is ready to harvest in late autumn. Additionally, there has been significant variation in flower induction timing across and within Cannabis cultivars.

Accordingly, there exists a need to consistently shorten the time to harvest for sun-grown Cannabis crops while maintaining any desired traits and yield.

### BRIEF DESCRIPTION OF THE DRAWINGS

FIG. 1 is a block diagram of example breeding results for day-neutral and short-day Cannabis plants, in accordance with the present disclosure.

### DETAILED DESCRIPTION

Disclosed herein are systems and methods for producing complex hybrids of Cannabis plants that begin flowering when exposed to a light cycle including about 540 minutes (9 hours) of uninterrupted darkness. As explained in detail below, it has been found that crossing homozygous varieties of short-day Cannabis sativa with homozygous varieties of day-neutral Cannabis ruderalis results in consistent early flowering short-day Cannabis hybrid progenies.

This type of crossbreeding of Cannabis plants has not been found to work with varieties of mulberry and hops plants, which are the closest relatives to Cannabis plants, found within the Moraceae and Cannabaceae families.

Plant species in the genus, Cannabis, are annual plants that are wind-pollinated to produce seeds that germinate the following year. Cannabis plants are dicotyledons that bear fruit in the form of achenes, which consist of one seed protected by two cotyledons or bracts (i.e., embryonic leaves) as well as energy rich nutritional proteins with all the essential amino acids.

Cannabis plant species are dioecious, meaning that staminate plants with a male sex chromosome (i.e., XY) have male flowers containing microgametophytes within the pollen, and pistillate plants with only female sex chromosomes (i.e., XX) have female flowers containing megagametophytes within the ovules. Hermaphroditic plants and flowers are also possible in monoecious phenotypes, although they are generally sterile. Morphological differences for visually distinguishing between male and female plants develop during the reproductive stage.

The diurnal light cycle and/or exposure to low levels of carbon monoxide may change the gender expression of a plant. Feminized seeds may also be produced by treating isolated portions of female plants with hormones or silver thiosulphate to induce pollen formation.

The life cycle of Cannabis plants includes germination/emergence, vegetative growth, reproductive stages, in which flowers and seeds are formed, and finally, senescence. The time for maturation may vary from about 2 to 10 months, but naturally, the time from seed to harvest is about 8 months. However, artificial indoor growing operations can speed the life cycle of Cannabis plants to just 90 days by boosting light exposure and tightly controlling the timing of the required photoperiods (discussed in detail below).

Cannabis seeds mostly lack dormancy mechanisms and germinate without requiring any pre-treating or winterizing. Weights range from about 2 to 70 grams per 1,000 seeds. When placed in viable growth conditions, Cannabis seeds germinate in about 1-19 days.

The stages of vegetative growth include time as a juvenile (basic vegetative phase) and a photosensitive phase, lasting until the development of flowers. Vegetative growth may last for about 2-20 weeks, during which growth increases in response to temperature and increasing light exposure, and plants may be grown to their desired size. After the juvenile stage of about 1-8 weeks, plants require at least 12 hours of light before flowering may begin about 1-12 weeks later. Exposing the plants in the photosensitive phase to a critical photoperiod (e.g., about 14-16 hours of light) begins flower development. In general, about 18-20 hours of light per day during the vegetative growth stage has been shown to produce the highest yields for some Cannabis plant varieties. Interrupting the continuity of just one night or darkness period during the photosensitive phase of the plant can delay or disrupt flower maturation. Whereas exposure to just one or two periods of short days (long nights) may induce flowering. In day-neutral (autoflowering) plants, entering the flowering stage may be irreversible.

Typically, sun-grown Cannabis plants flower between June and September, depending on the latitude. The flowering stage may range from about 5 to 16 weeks, depending on the genetics and environment. After the initially developed flowers that have been pollinated produce their fruit and seeds, pistillate plants may continue to produce additional flowers while staminate plants die. Colder weather eventually kills pistillate plants unless they are grown indoors or artificially induced into a vegetative state.

After being grown, Cannabis plants may be harvested at full flowering or at the end of flowering for their fiber, seeds, or cannabinoids. Indicators that plant flowers are ready for harvest may include stigmas changing color or disappearing.

Photoperiod refers to a plant's response to the amount of light and darkness, to which it is exposed. Depending on the genetics, light exposure events will trigger transcription factors, which activate flowering genes within plants. Without being bound to theory, it is thought that light-sensitive chromophore moieties within a Cannabis plant's cytochrome protein molecules change form or state depending on the energy or wavelength of the light absorbed. The changed form of the cytochrome protein may then signal the relay pathways or initiate the biochemical reactions for the activation of flowering genes. Additionally, genetically determined biochemical oscillators responsible for the circadian rhythms within a plant may dictate the entrainment or synchronization of the plant's internal clock with its environmental light-dark cycle. Phosphorylation or other reactions affecting the regulation of gene expression through the binding rates of pseudo-response regulators may peak at different times throughout the day (i.e., 24-hour period), such as at night or during an interval of uninterrupted darkness, in accordance with the negative feedback loop of the plant's biological clock.

For example, short-day or long-night plants, as obligate photoperiodic plants, will only begin flowering once the sunlight hours are reduced to a certain number, based on the seasonal changes of the earth's orbit or artificial replication thereof. Typically, short-day plants will flower when the day is less than 12 hours (i.e., the night is longer than 12 hours) regardless of plant age or size. In indoor growing operations, this photosensitivity allows for a precisely tailored plant cycle for continuous growing seasons with the stages of development being artificially controlled. Additionally, when outdoors, short-day plants can be fooled into flowering early (i.e., outside of the natural seasonal schedule) by being covered for at least 12 hours in a 24-hour period. Similarly, if exposed to more than 12 hours of light in a 24-hour period, short-day plants will not flower, so flowering may be delayed and/or a plant may be kept in a perpetual vegetative state (e.g., as a mother plant for clones and/or seeds).

Autoflowering or day-neutral plants, by contrast, will flower regardless of day or night length, based on various factors including plant maturity, total amount of light exposure, angle of the sun, degree-days, and root system containment. Indoor growing operations can therefore cause day-neutral plants to flower quickly or early based on the amount of light exposure, even running grow lights constantly. Conversely, this means that day-neutral plants may not be preserved in a vegetative state and will flower no matter if placed in perpetual darkness or light.

All plants within the genus, Cannabis, are obligate photoperiodic plants, and more specifically, short-day plants, except for Cannabis ruderalis, which is day-neutral. The variety Cannabis ruderalis Janischewsky may be synonymous with C. sativa var. ruderalis Janisch, C. sativa subsp. sativa var. spontanea Vavilov, C. sativa var. spontanea Czernj., and C. sativa subsp. spontanea Serebr. Most Cannabis sativa plants flower when the length of continuous darkness exceeds about 10-12 hours per 24-hour period or when daylight lengths only last about 12-14 hours. Cannabis sativa seeds are generally planted between March and May and harvested about 6-8 months later, between September and November. Short-day Cannabis sativa plants may be induced into a vegetative state using a 16- to 24-hour lighting cycle. Day-neutral Cannabis plants will flower based on the maturity of the plant after germination and do not depend on a change in photoperiod, however, they tend to flower earlier with longer days.

Despite the autoflowering property of day-neutral Cannabis ruderalis plants, it tends to be a less desired variety of Cannabis plant due to its small yield and low cannabinoid content. Some breeders have produced autoflowering hybrids of Cannabis ruderalis and Cannabis sativa or Cannabis indica with a vegetative growth stage of only 6 weeks or about 45 days—e.g., strains such as Low Ryder and Auto AK-47. However, unlike the carefully selected clone mother plants of sativa or indica strains, autoflowering hybrids may not be kept in a vegetative state so that its desirable properties may be cloned from clippings. Further, within industrial fiber hemp growing, early flowering is considered undesirable due to its effect of halting stalk growth, thus limiting yield.

Cannabis ruderalis may be further differentiated from other Cannabis varieties by the morphological differences in its smaller achenes—formed with a constricted base, swollen abscission zone (eliosome), and mottled perianth, adherent to the achene—that separate from the plant after flowering and have a wider distribution in terms of germination. Cannabis ruderalis plants are shorter (typically less than 2 feet tall) and unbranched, but boast a higher resistance to cold and a faster maturation. For example, Finola is a strain of Finnish seed hemp that is early maturing (i.e., has a relatively short juvenile phase of about 13 days). The seeds of Cannabis ruderalis can survive freezing and in just 10 weeks, can complete their entire life cycle.

Cannabis plants uniquely contain C₂₁ or C₂₂ terpenophenolic chemical compounds known as cannabinoids—specifically, phytocannabinoids that naturally occur within the plant itself. Many Cannabis crops are harvested specifically to collect these cannabinoids for various downstream uses, so often plant varieties are bred to maximize their total cannabinoid yield. The phytocannabinoids within a Cannabis plant are secondary metabolites synthesized within glandular trichome cells and may include cannabigerolic acid (CBGA), which can be converted into cannabichromenic acid (CBCA), cannabidiolic acid (CBDA), and/or tetrahydrocannabinolic acid (THCA) depending on the type of enzymes present in the plant according to its genetics. Specifically, the oxidoreduction and cyclization of CBGA catalyzed by THCA and CBDA synthases provides the synthesis of THCA and CBDA. The phytocannabinoid content (e.g., THCA/CBDA ratio) resulting from the plant's genetics allow for classification of Cannabis plant types by discrete chemical phenotype or chemotype, as shown in Table A below.

TABLE A
CANNABIS PLANT TYPE CATEGORIZATION

| Cannabis | |
|---|---|
| Chemotype | Phytocannabinoid Content Description |
| Type I | THCA dominant |
| Type II | Substantially equal parts CBDA and THCA |
| Type III | CBDA dominant |
| Type IV | CBGA dominant |
| Type V | Cannabinoid free (i.e., containing terpenes but no |
| | cannabinoids) |

The overall THCA/CBDA ratio is thought to be genetically predetermined and thus, does not vary significantly throughout the life of the plant.

Additionally, the expression of the CBGA pure or dominant chemotype for Type IV plants may have resulted from self-fertilization or inbreeding within monoecious or hermaphroditic plants creating a fixed, mutated B₀ allele. The expression of the cannabinoid-free chemotype for Type V plants may be due to null genotypes at an A locus. Further chemotype expressions are possible, such as CBCA synthase encoding with the B_{C} allele, for example. The frequency of the THCA synthase allele (B_{T}) and plants with propyl sidechain cannabinoids have been found to be higher within Cannabis indica varieties than in varieties of Cannabis sativa and Cannabis ruderalis. Often, however, the different chemotypes or varieties of Cannabis plants are crossbred, leading to interesting new traits but increasing the heterozygosity of the resulting progenies. The resulting plants grown from the seeds created from crossbreeding two parent plants are referred to as F₁ progeny plants. F₁ progenies often have reduced homozygosity, causing instability in their expressed traits.

The homozygosity (i.e., genetic variation) of Cannabis plants may be measured in terms of the amount of polymorphism observed in scoring randomly amplified polymorphic DNA markers and/or performing amplified fragment length polymorphism analyses. Moreover, the bulk segregant analysis strategy for finding molecular markers may be used when F₂ progenies (from interbreeding F₁ individuals) exhibit clear cut segregation.

Inbreeding plants involves some form of self-crossing or asexual propagation (e.g., cloning by self-pollination or clipping), which may reduce the amount of heterozygosity within the genetics. For example, experiments have shown that doubly inbred plants (i.e., S₂ progenies) exhibit less genetic variation as compared to non-inbred plants.

If self-crossed plants (i.e., S₁ or S₂ progenies) are crossed such that the resulting plants (i.e., F₁ progenies) segregate into distinct phenotypes, it indicates that the self-crossed parent plants were still heterozygous at the relevant loci. In general, the CBDA content within heterozygous F₁ progenies of crossed pure homozygous chemotypes (i.e., Types I and III) is higher than that of Type III parent plants derived from fiber strains with lower inflorescence density and total cannabinoid content. Further, deviations from a strictly even dispersal within the tripartite cannabinoid ratio distribution model among F₂ progenies may indicate a natural preference away from the Type III chemotype due to a recessive and unfavorable factor that corresponds to the B_{D} allele, evidenced in the significantly reduced fertility of pure CBDA plants expressed during embryogenesis.

Industrial Cannabis plants, those with less than 0.3% THCA, have an innumerable variety of uses, including hemp products and CBDA oil.

In addition to cannabinoid content, the rate of maturation of a plant may be a highly valued characteristic. Although the more rapid maturation of Cannabis ruderalis is undesirable for hemp fiber production due to its low yield, faster time to flowering may be coveted in other applications. Specimens of Cannabis sativa that have been shown to flower earlier are retained and propagated asexually. However, this method of cloning a single prized specimen is unable to be scaled up for multiacre industrial outdoor growing operations.

Indoor and other smaller growing operations rely on mother plants to provide genetic clones through clippings. In general, the relative heterozygosity of a clone should not matter, because the resulting asexually reproduced plant should have sufficiently the same characteristics as the mother plant, provided that the mother plant has been carefully bred and selected for stable asexual propagation. However, as mentioned above, it is very difficult to scale up this type of breeding, so outdoor and/or multiacre growing operations use seeds created from sexual reproduction within plants.

The results of the experiments described in detail below illustrate that it is possible to crossbreed different phenotypes of Cannabis to produce seeds that will consistently grow into early flowering short-day plants. As used here, the term “consistent” means that the seeds produce early flowering, short-day plants across most growing conditions, including latitude, which affects periods of light.

Cross-hybridization of Cannabis biotypes, such as between Cannabis sativa, Cannabis indica, and/or Cannabis ruderalis, has led to more heterozygosity among hybrid plants. Genetic heterozygosity or heterogeneity, by its nature, does not result in trait stable seed stock due to the tendencies of the combined genes to self-segregate into tripartite or other phenotype distributions, as described above. Industrial hemp breeding and outdoor multiacre growing operations require stabilization or fixation of certain traits within the seed stock, and thus, reduced heterozygosity.

The method may include back-crossing or self-crossing varieties to produce purer species (i.e., with reduced heterozygosity) from hybridized strains. Reducing heterozygosity may involve inbreeding female plants until a fixed homozygous phenotype is achieved. For example, Cannabis sativa plants may be bred among themselves (e.g., through interbreeding or self-breeding) until producing a set of plants that are each sufficiently homozygous. Additionally or alternatively, Cannabis ruderalis plants may be similarly bred among themselves until a sufficient level of homozygosity is reached. Reducing heterozygosity may require several generations of breeding depending on the level of homozygosity or purity desired. The process of reducing homozygosity may result in the creation of an inbred line that produces plants with minimized differences between each other. As described above, homozygosity or homology may be measured through genetic testing and/or other method of morphological, varietal, biotypical, or phenotypical identification.

Self-fertilization, self-pollination, or self-crossing of plants may be performed by hand-pollinating female flowers with pollen from induced male flowers on the same plant. The male flowers may be induced by the application of an aqueous solution of silver nitrate (AgNO₃) to the growing shoot tip of the female plant, in accordance with the method disclosed in Ram et al., “Induction of Fertile Male Flowers in Genetically Female Cannabis sativa Plants by Silver Nitrate and Silver Thiosulphate Anionic Complex”, Theor. Appl. Genet. 62, 369-375 (1982), which is herein incorporated by reference. The seeds bore from the self-pollination may produce only pistillate female plants due to their genetics containing only female sex chromosomes.

Other methods for self-pollination, whether through inducing fertile male flowers on pistillate plants (e.g., using colloidal silver, gibberellic acid, or Rodelization), feminization of staminate plants (e.g., using ethephon), or alternative means (e.g., irradiation, streptovaricin treatment), are also possible.

Once a desired homozygosity has been reached and/or homologous phenotypes or specimens have been selected, a short-day Cannabis plant may be crossbred with a day-neutral Cannabis plant to produce an F₁ generation of seeds. The short-day Cannabis plant may be a sufficiently homozygous Cannabis sativa plant, selected based on cannabinoid content, for example. The day-neutral Cannabis plant may be a sufficiently homozygous Cannabis ruderalis plant, selected based on maturation time or yield, for example.

Cross-pollination or crossbreeding of two parent plants may also be performed by chemically inducing male flowers on one of the genetically female plants in order to ensure all genetically female progeny. However, it is not believed that the particular sex chromosomes play any role in genetically defining the early flowering trait, so male plants may also be used in any of the methods disclosed herein. Advantageously on a broader scale of outdoor field crops, utilizing feminized seed—such as from pistillate female plants with induced male flowers—reduces the risk of pollination and contamination between neighboring farms.

Based on the heterogenous nature of the parent short-day and day-neutral plants at the relevant gene loci coding for photoperiod and/or growth timing, the F₁ generation of seeds may produce consistently early flowering short-day plants. The F₁ plants may flower within 2.5 weeks of each other. Plants of this F₁ generation may be further self-crossed and/or back-crossed to reintroduce day-neutral or other desired traits, such as cannabinoid or terpine content, described below. For example, once the day-neutral property is reintroduced into the resulting progenies, they may again be crossbred with a selected short-day Cannabis plant to produce another F₁ generation of seeds that will grow into early flowering short-day plants.

In addition to cannabinoids, Cannabis plants also include aromatic secondary metabolites, such as flavonoids and terpenoids or terpenes. Such terpenoids (e.g., mono-, di-, and sesquiterpene oils) or flavonoids may include α-bisabolol, borneol, isoborneol, menthol, nerol, camphene, camphor, Δ³-carene, α-cedrene, β-eudesmol, eudesmol, fenchol, geraniol, β-myrcene, myrcene, α-terpinene, α-terpineol, α-terpinolene, terpinolene, α-phelladerene, α-pinene, β-pinene, pinene, sabinene, α-humulene, humulene, β-caryophyllene, caryophyllene oxide, trans-caryophyllene, cis-ocimene, trans-ocimene, geranyl diphosphate, farnesol, leucosceptrine, squalene, limonene, phytol, guaiol, and linalool, for example.

It has been found that each Cannabis biotype (e.g., Cannabis sativa, Cannabis indica, Cannabis ruderalis) has commonalities among the terpene profiles of its strains. For example, Cannabis sativa strains may be called Diesel due to the higher levels of terpenes such as humulene and/or β-caryophyllene. The interaction of specific terpenes with the receptors in mammalian brains and bodies may affect the binding of both endocannabinoids and phytocannabinoids. Thus, selection for a particular terpene profile within a plant may be application specific.

As mentioned above, Cannabis plants of the F₁ generation may be further selected for breeding based on their organoleptic appeal due to resin, cannabinoid, and/or terpene levels. Such categories of aromatic selection may include, but are not limited to, berry, citrus, pine, lemon, and/or diesel.

The F₁ generation of Cannabis plants may further be selected for seed and/or fiber yield and/or quality, depending on the industrial application.

Advantageously, crosses between Cannabis sativa and Cannabis ruderalis may have increased structural integrity based on woodier, thicker stems, and even absence of a primary stem, which results in a plant less susceptible to wind damages. Additionally, the disclosed methods may allow consistently early flowering progenies to be planted from seed rather than from clone, which enables industrial scale farming of the Cannabis crops while making efficient use of field time.

### Examples

Under the industrial hemp research legalization within Section 7606 of the Agricultural Act of 2014, the following experiments were conducted as part of Oregon's agricultural pilot program for the growth, cultivation, and marketing of industrial hemp. All plantings occurred within a week of June 1.

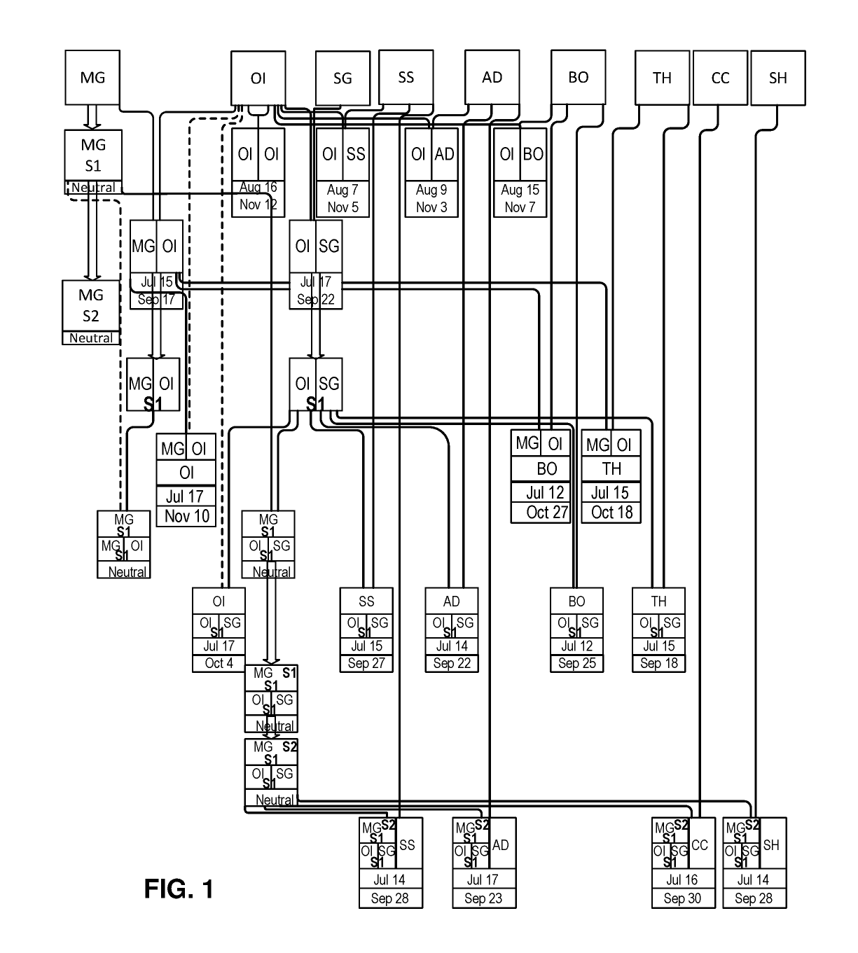

Several different Cannabis plants were self-crossed, back-crossed, and/or crossed, then studied for their photoperiodism and time to harvest at a latitude of about 44.45° N. The plants used in the experiments were all genetically female. Beginning with a Type II day-neutral Cannabis plant, the Type II plant was self-pollinated and its progeny sifted through until finding a Type III day-neutral plant. The variety of parent Cannabis strains tested include two day-neutral strains (MG and SG) and seven short-day strains (OI, SS, AD, BO, TH, CC, and SH). FIG. 1 illustrates the breeding methods and results for these day-neutral and short-day Cannabis varieties including the dates of flowering and harvesting.

Self-crossing was performed in accordance with the silver nitrate method of inducing male flowers on a pistillate plant described above.

Crosses between OI and the other short-day Cannabis sativa strains resulted in short-day progeny with (1) OI/OI F1 plants flowering August 16^{th} and were ready for harvest November 12^{th}, (2) OI/SS F1 plants flowering August 7^{th} and were ready for harvest November 5^{th}, (3) OI/AD F1 plants flowering August 9^{th} and were ready for harvest November 3^{rd}, and (4) OI/BO F1 plants flowering August 15^{th} and were ready for harvest November 7^{th}. These results were in accordance with typical flowering and harvest times for short-day Cannabis sativa varieties.

MG, a day-neutral Cannabis plant, was self-crossed to propagate MG_{S1} progeny, which was self-crossed again to propagate MG_{S2}. Both MG_{S1} and MG_{S2} exhibited day-neutral characteristics, as would be expected through inheriting the genetics of only MG.

MG was further crossed with OI, a short-day Cannabis plant. The resulting MG/OI F1 progeny hybrids were short-day Cannabis plants that began flowering July 15^{th} and were ready for harvest September 17^{th}.

The MG/OI F1 hybrids were crossed again with three different short-day Cannabis sativa varieties. The first was a back-cross of the MG/OI F1 progeny with OI, resulting in short-day plants that flowered July 17^{th}, but were not ready for harvest until November 10^{th}, similar to the crosses between pure Cannabis sativa varieties. The other two crosses of the MG/OI F1 hybrid progeny with short-day varieties were (1) a cross with BO, resulting in short-day plants that flowered July 12^{th}, but were not ready for harvest until October 27^{th}, and (2) a cross with TH, resulting in short-day plants that flowered July 15^{th}, but were not ready for harvest until October 18^{th}. Based on these results, it seems that back-crossing a short-day/day-neutral hybrid with the original short-day Cannabis sativa variety is more likely to reverse the earlier harvest characteristics of the hybrid than if the hybrid is crossed with another short-day Cannabis sativa variety.

The MG/OI F1 hybrids were self-crossed to produce MG/OI_{S1} progenies, which were then crossed with MG_{S1}. The resulting progeny of MG/OI_{S1} and MG_{S1} were day-neutral, likely due to the reintroduction of the MG plant's genetics.

SG, another day-neutral Cannabis plant, was crossed with OI, a short-day Cannabis plant. The resulting SG/OI F1 hybrid produced seeds that began flowering July 7^{th} and were ready for harvest September 22^{nd}. The SG/OI F1 hybrids were self-crossed to produce a SG/OI_{S1} generation. The SG/OI_{S1} hybrid plants were crossed with the day-neutral MG_{S1} plants, which resulted in day-neutral progeny SG/OI/MG that was self-crossed for two generations into the day-neutral SG/OI/MG_{S1} and SG/OI/MG_{S2} progenies.

The SG/OI_{S1} hybrid plants were also crossed with five short-day Cannabis sativa varieties. The first cross reintroduced OI to the hybrid results, which were short-day plants that flowered July 17^{th}, but were not ready for harvest until October 4^{th}. The other four crosses for SG/OI_{S1} hybrids with short-day plants were (1) a cross with SS, resulting in short-day plants that flowered July 15^{th} and were ready for harvest September 27^{th}, (2) a cross with AD, resulting in short-day plants that flowered July 14^{th} and were ready for harvest September 22^{nd}, (3) a cross with BO, resulting in short-day plants that flowered July 12^{th} and were ready for harvest September 25^{th}, and (4) a cross with TH, resulting in short-day plants that flowered July 15^{th} and were ready for harvest September 18^{th}.

Finally, the day-neutral SG/OI/MG_{S2} plants were crossed with four short-day Cannabis sativa varieties, resulting in (1) a short-day hybrid with SS that flowered July 14^{th} and was ready for harvest September 28^{th}, (2) a short-day hybrid with AD that flowered July 17^{th} and was ready for harvest September 23^{rd}, (3) a short-day hybrid with CC that flowered July 16^{th} and was ready for harvest September 30^{th}, and (4) a short-day hybrid with SH that flowered July 14^{th} and was ready for harvest September 28^{th}. As can be seen above, the early flowering plants flowered in July, and were ready for harvest by mid-September, while the plants that follow the natural season would not flower until August and were not ready for harvest until November.

Seeds resulting from these methods have been grown in regions of differing latitude, meaning the regions have different light periods and growing conditions. All plants reach peak maturity within 2.5 weeks of each other, with an average maturity of mid-September for plants resulting from the seeds planted at or near the same time.

### DEPOSIT INFORMATION

A deposit of the seeds of the cannabis varieties of the present invention, is maintained by Jack Hempicine, LLC, 7744 NW Mint Avenue, Albany, Oreg., 97321.

In addition, seeds and/or a sample for one of more varieties of this invention have been or will be deposited with an International Depository Authority as established under the Budapest Treaty according to 37 CFR 1.803(a)(1), such as the American Type Culture Collection (ATCC) in Manassas, Va., and the Agricultural Research Service Culture Collection (NRRL, the acronym is based upon an older name), in Peoria, Ill., or a depository recognized to be suitable by the United States Patent and Trademark Office under 37 CFR 1.803(a)(2).

To satisfy the enablement requirements of 35 USC 112, and to certify that the deposit of the seeds and/or a sample of the present invention meets the criteria set forth in 37 CFR 1.801-1.809 and the Manual of Patent Examining Procedure (MPEP) 2402-2411.05, Applicants hereby make the following statements regarding the deposited seed varieties:

If the deposit is made under the terms of the Budapest Treaty, the instant invention will be irrevocably and with restriction released to the public upon granting of a patent.

If a deposit is made not under the terms of the Budapest Treaty, Applicant(s) provides assurance of compliance by the following statements:

1. During the pendency of this application, access to the invention will be afforded to the Commission upon request;

2. All restrictions on availability to the public will be irrevocably removed upon granting of the patent under conditions specified under 37 CFR 1.808;

3. The deposit will be maintained in a public repository for a period of 30 years or 5 years after the last request or for the effective life of the patent, whichever is longer;

4. A test of the viability of the biological material at the time of deposit will be conducted by the public depository under 37 CFR 1.807; and

5. The deposit will be replaced it if should ever become unavailable.

Access to this deposit will be available during the pendency of this application to persons determined by the Commissioner of Patents and Trademarks to be entitled thereto under 37 CFR 1.14 and 35 USC 122. Upon granting of any claims in this application, all restriction on the availability to the public of the variety will be irrevocably removed by affording access to a deposit of at least 2,500 seeds of the same variety with the depository.

The previously described versions of the disclosed subject matter have many advantages that were either described or would be apparent to a person of ordinary skill. Even so, all of these advantages or features are not required in all versions of the disclosed apparatus, systems, or methods.

Additionally, this written description makes reference to particular features. It is to be understood that the disclosure in this specification includes all possible combinations of those particular features. For example, where a particular feature is disclosed in the context of a particular aspect, that feature can also be used, to the extent possible, in the context of other aspects.

Also, when reference is made in this application to a method having two or more defined steps or operations, the defined steps or operations can be carried out in any order or simultaneously, unless the context excludes those possibilities.

Although specific aspects of the invention have been illustrated and described for purposes of illustration, it will be understood that various modifications may be made without departing from the spirit and scope of the invention. Accordingly, the invention should not be limited except as by the appended claims.

The aspects of the present disclosure are susceptible to various modifications and alternative forms. Specific aspects have been shown by way of example in the drawings and are described in detail herein. However, it should be noted that the examples disclosed herein are presented for the purposes of clarity of discussion and are not intended to limit the scope of the general concepts disclosed to the specific aspects described herein unless expressly limited. As such, the present disclosure is intended to cover all modifications, equivalents, and alternatives of the described aspects in light of the attached drawings and claims.

References in the specification to aspect, example, etc., indicate that the described item may include a particular feature, structure, or characteristic. However, every disclosed aspect may or may not necessarily include that particular feature, structure, or characteristic. Moreover, such phrases are not necessarily referring to the same aspect unless specifically noted. Further, when a particular feature, structure, or characteristic is described in connection with a particular aspect, such feature, structure, or characteristic can be employed in connection with another disclosed aspect whether or not such feature is explicitly described in conjunction with such other disclosed aspect.

What is claimed is:

1. A method of developing seeds for consistent early flowering short-day Cannabis plants, the method comprising:
crossbreeding a short-day Cannabis plant with a day-neutral Cannabis plant; and
collecting the seeds produced through crossbreeding.

2. The method as claimed in of claim 1, wherein the short-day Cannabis plant is Cannabis sativa.

3. The method as claimed in claim 1, wherein the day-neutral Cannabis plant is Cannabis ruderalis.

4. The method as claimed in claim 1, further comprising selecting the short-day Cannabis plant based on at least one of resin levels, terpine profile, cannabinoid levels, and structure.

5. The method as claimed in claim 1, further comprising selecting the day-neutral Cannabis plant based on at least one of resin levels, terpine profile, cannabinoid levels, and structure.

6. The method as claimed in claim 1, further comprising inbreeding the short-day Cannabis plant prior to the crossbreeding.

7. The method as claimed in claim 6, wherein inbreeding includes:
inducing staminate flowers on a pistillate plant; and
pollinating pistillate flowers on the pistillate plant with pollen from the induced staminate flowers.

8. The method as claimed in claim 6, further comprising selecting the short-day Cannabis plant for inbreeding based on at least one of resin levels, terpine profile, cannabinoid levels, and structure.

9. The method as claimed in claim 6, wherein inbreeding the short-day Cannabis plant includes homogenizing the initial Cannabis plant for at least 3 generations.

10. The method as claimed in claim 1, further comprising inbreeding the day-neutral Cannabis plant prior to the crossbreeding.

11. The method as claimed in claim 10, wherein inbreeding includes:
inducing staminate flowers on a pistillate plant; and
pollinating pistillate flowers on the pistillate plant with pollen from the induced staminate flowers.

12. The method as claimed in claim 10, further comprising selecting the day-neutral Cannabis plant for inbreeding based on at least one of resin levels, terpine profile, cannabinoid levels, and structure.

13. The method as claimed in claim 10, wherein inbreeding the day-neutral Cannabis plant includes homogenizing the initial Cannabis plant for at least 3 generations.

14. The method as claimed in claim 1, wherein crossbreeding includes:
inducing staminate flowers on a pistillate plant; and
pollinating a second pistillate plant with pollen from the induced staminate flowers.

15. A Cannabis seed, developed from the crossbreeding of a short-day and a day-neutral Cannabis plant, comprising:
early flowering characteristics, wherein the Cannabis plant grown from the seed will flower when exposed to a critical photoperiod that includes about 540 minutes of uninterrupted darkness.

16. The Cannabis seed as claimed in claim 15, wherein the short-day Cannabis plant is Cannabis sativa.

17. The Cannabis seed as claimed in claim 15, wherein the day-neutral Cannabis plant is Cannabis ruderalis.

18. A Cannabis seed obtained by crossing:
a first Cannabis plant that is transformed preferentially to have a first homozygosity level and to express short-day characteristics, whereby flowering depends on a change in photoperiod; and
a second Cannabis plant that is transformed preferentially to have a second homozygosity level and to express day-neutral characteristics, whereby flowering does not depend on a change in photoperiod.

19. The Cannabis seed as claimed in claim 18, wherein the first and second homozygosity levels are measured by genetic testing.

20. A method of producing an early flowering Cannabis plant, the method comprising:
crossing a day-neutral Cannabis ruderalis plant with a short-day Cannabis sativa plant;
recovering the resultant F₁ hybrid Cannabis seed;
planting the resultant F₁ hybrid Cannabis seed and growing into plants; and
selecting resultant Cannabis plant from the planting having a desired trait.
